(19) United States
(12) Patent Application Publication
(10) Pub. No.: US 2022/0160692 A1
(43) Pub. Date: May 26, 2022

(54) USE OF SK2 INHIBITORS IN COMBINATION WITH IMMUNE CHECKPOINT BLOCKADE THERAPY FOR THE TREATMENT OF CANCER

(71) Applicants: INSERM (Institut National de la Santé et de la Recherche Médicale), Paris (FR); Université Paul Sabatier Toulouse III, Toulouse (FR); Centre Hospitalier Universitaire de Toulouse, Toulouse (FR)

(72) Inventors: Alexandre GHENASSIA, Toulouse Cedex 1 (FR); Nathalie ANDRIEU-ABADIE, Toulouse (FR); Bruno SEGUI, Toulouse Cedex 1 (FR); Thierry LEVADE, Toulouse (FR)

(21) Appl. No.: 17/602,880

(22) PCT Filed: Apr. 8, 2020
(86) PCT No.: PCT/EP2020/059984

§ 371 Date: Oct. 11, 2021

(30) Foreign Application Priority Data
Apr. 9, 2019 (EP) 19305461.6

(51) Int. Cl. A61K 31/4409; A61K 39/395; A61P 35/00

(52) U.S. Cl. CPC A61K 31/4409; A61P 35/00; A61K 39/3955

(57) ABSTRACT

Immune checkpoint blockade therapy is based on the inhibition of the tumor-mediated suppression of anticancer immune responses. However, the efficacy and effectiveness of said therapy vary greatly across individual patients and among different tumor types. A substantial unmet need is thus to identify novel targets that can enhance the therapeutic efficacy of the immune checkpoint blockade therapy. S1P is produced by sphingosine kinases (i.e. SK1 and SK2) that catalyze the phosphorylation of sphingosine to SIP. SK2 inhibitors were described as suitable for the treatment of cancer. However the role of SK2 in the immune tumor microenvironment has never been investigated. The inventors now showed that genetic deletion of SPHK2 leads to a delay in the melanoma tumor growth and an increase in tumor-infiltrating effector lymphocytes. In particular the increase of tumor-infiltrating effector lymphocytes in the tumor is associated with a decrease in the amount of tumor-infiltrating myeloid-derived suppressor cells. Moreover, the combination of SPHK2 deficiency with immune-checkpoint blockade leads to tumor rejection and increases survival rate. Accordingly, the present invention relates to use of SK2 inhibitors in combination with immune checkpoint blockade therapy for the treatment of cancer.

### FIELD OF THE INVENTION

The present invention is in the field of oncology and immunology.

### BACKGROUND OF THE INVENTION

Immune checkpoint blockade therapy is based on the inhibition of the tumor-mediated suppression of anticancer immune responses. T-cell activation is indeed regulated by the interplay of the stimulatory and inhibitory ligand receptor interactions between T cells, dendritic cells, tumor cells, and macrophages in the tumor microenvironment (TME), with tumor cells acting as critical mediators of immunosuppression. Owing to their roles as regulators of T-cell activation, these receptor ligand pairs are called ‘immune checkpoints’. Agents targeting these checkpoints have been identified as promising treatment options for patients with cancer. Immune-checkpoint inhibitors (ICIs) include, among others, monoclonal antibodies to the receptor cytotoxic T-lymphocyte antigen-4 (CTLA-4) expressed on T cells; programmed cell death protein 1 (PD-1), also expressed on T cells; or the PD-1 ligand (PD-L1), which is expressed by a variety of cell types, including some tumor cells. For instance, the anti-PD-1 antibodies nivolumab and pembrolizumab, and the anti-PD-L1 antibody atezolizumab, have shown marked therapeutic activity in various solid tumors and lymphomas, resulting in a number of regulatory approvals of these agents for the treatment of different malignancies. However, the efficacy and effectiveness of these therapies varies greatly across individual patients and among different tumor types. A substantial unmet need is thus to identify novel targets that can enhance the therapeutic efficacy of the immune checkpoint blockade therapy.

S1P is produced by sphingosine kinases (i.e. SK1 and SK2) that catalyze the phosphorylation of sphingosine to SIP. The SK type 1 isoform (SK1), which is overexpressed in numerous human tumors including melanoma, leads to increased levels of SIP. Moreover, SK1 inhibitors were described as suitable for enhancing the potency of the immune checkpoint inhibitors (WO2017129769). Moreover, SK2 inhibitors were described as suitable for the treatment of cancer (Lewis, C. S., Voelkel-Johnson, C. and Smith, C. D., 2018. Targeting sphingosine kinases for the treatment of cancer. In Advances in cancer research (Vol. 140, pp. 295-325). Academic Press).

### SUMMARY OF THE INVENTION

As defined by the claims, the present invention relates to use of SK2 inhibitors in combination with immune checkpoint blockade therapy for the treatment of cancer.

### DETAILED DESCRIPTION OF THE INVENTION

The first object of the present invention relates to a method of increasing the amount of tumor infiltrating cytotoxic T lymphocytes cells in a patient suffering from cancer comprising administering to the patient a therapeutically effective amount of a SK2 inhibitor.

As used herein, the term “cytotoxic T lymphocyte” or “CTL” has its general meaning in the art and refers to a subset of T cells which express CD8 on their surface. CD8 antigens are members of the immunoglobulin supergene family and are associative recognition elements in major histocompatibility complex class I-restricted interactions. They are WIC class I-restricted, and function as cytotoxic T cells. Cytotoxic T lymphocytes are also called, CD8+ T cells, T-killer cells, cytolytic T cells, or killer T cells. As used herein, the term “tumor-infiltrating cytotoxic T lymphocyte” refers to the pool of cytotoxic T lymphocytes of the patient that have left the blood stream and have migrated into a tumor. For example, the SK2 inhibitor of the present invention has the ability to increase the amount of tumor-infiltrating cytotoxic T lymphocytes cells by more than about 10%, preferably with at least about 15%, at least about 20%, at least about 25%, or more. In some embodiments, the SK2 inhibitor is particularly suitable for increasing the amount of CD8⁺PD1⁺ T cells. Typically said tumor-infiltration of CTL cells is determined by any convention method in the art. For example, said determination comprises quantifying the density of CD8⁺ T cells or CD8⁺ PD1⁺ T cells in a tumor sample obtained from the patient.

A further object of the present invention relates to a method of reducing the amount of tumor-infiltrating myeloid-derived suppressor cells in a patient suffering from cancer comprising administering to the patient a therapeutically effective amount of a SK2 inhibitor.

As used herein, the term “myeloid-derived suppressor cells” (MDSCs) refers to cells that exist in the microenvironment of a tumor, are immunosuppressive, and are of myeloid lineage. Myeloid-derived suppressor cells (MDSCs) are known to enhance immunosuppression in the tumor environment by suppressing such cells as T cells, NK cells, DC, macrophages, and NKT cells. Thus, MDSCs can promote tumor growth, angiogenesis, and metastasis. The abundance of these cells in the tumor environment correlates negatively with cancer patient survival. Thus, therapies that reduce the amount of MDSCs are desirable. Human MDSCs are characterized by at least the expression of the cell markers CD11b and CD33. Human MDSCs may also express the markers CD15 and/or CD14. For example, the SK2 inhibitor of the present invention has the ability to reduce the amount of tumor-infiltrating MDSCs at least about 25%, or more. Typically said tumor-infiltration of MDSCs determined by any convention method in the art. For example, said determination comprises quantifying the density of CD11b⁺CD33⁺ cells in a tumor sample obtained from the patient.

As used herein, the term “tumor tissue sample” means any tumor tissue-derived sample from the patient. Said tissue sample is obtained for the purpose of the in vitro evaluation. In some embodiments, the tumor sample may result from the tumor resected from the patient. In some embodiments, the tumor sample may result from a biopsy performed in the primary tumor of the patient or performed in metastatic sample distant from the primary tumor of the patient. For example, an endoscopical biopsy performed in the bowel of the patient affected by a colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, through flow cytometry or mass cytometry methods and through methods of gene or protein expression analysis, including genomic and proteomic analysis. The tumor tissue sample can, of course, be patented to a variety of well-known post-collection preparative and storage techniques (e.g., fixation, storage, freezing, etc.). The sample can be fresh, frozen, fixed (e.g., formalin fixed), or embedded (e.g., paraffin embedded).

In some embodiments, the quantification of density of CTL and/or MDSCs is determined by immunohistochemistry (IHC).

For example, the quantification of the density the cells is performed by contacting the tumor tissue sample with a binding partner (e.g. an antibody) specific for a cell surface marker of said cells. Typically, the quantification of density of CD8+ T cells is performed by contacting the tumor tissue sample with a binding partner (e.g. an antibody) specific for CD8. Typically, the quantification of density of CD8+PD1+ T cells is performed by contacting the tumor tissue sample with a binding partner (e.g. an antibody) specific for CD8 and a binding partner (e.g. an antibody) specific for PD1. Typically, the quantification of density of MDSCs is performed by contacting the tumor tissue sample with a binding partner (e.g. an antibody) specific for CD11b and a binding partner (e.g. an antibody) specific for CD33 Typically, the density is expressed as the number of these cells that are counted per one unit of surface area of tissue sample, e.g. as the number of cells that are counted per cm² or mm² of surface area of tumor tissue sample. In some embodiments, the density of cells may also be expressed as the number of cells per one volume unit of sample, e.g. as the number of cells per cm3 of tumor tissue sample. In some embodiments, the density of cells may also consist of the percentage of the specific cells per total cells (set at 100%).

Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labeling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled.

Typically, the resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale.

Thus, in some embodiments, the IHC method consists in i) providing one or more immunostained slices of tissue section obtained by an automated slide-staining system by using a binding partner capable of selectively interacting with the marker (e.g. an antibody as above described), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

Multiplex tissue analysis techniques are particularly useful for quantifying several proteins in the tumor tissue sample (e.g CD8 and PD1). Such techniques should permit at least five, or at least ten or more biomarkers to be measured from a single tumor tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.).

In some embodiments, the L-IHC method can be performed on any of a variety of tissue samples, whether fresh or preserved. The samples included core needle biopsies that were routinely fixed in 10% normal buffered formalin and processed in the pathology department. Standard five μtri thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10μη thick coated polymer backbone with 0.4μη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.). That technique is described in U.S. Pat. No. 7,473,532.

In some embodiments, the method of the present invention utilized the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the patient, ii) TMA samples are then stained with anti-antibodies having specificity for markers of interest, iii) the TMA slide is further stained with an epithelial cell marker to assist in automated segmentation of tumor and stroma, iv) the TMA slide is then scanned using a multispectral imaging system, v) the scanned images are processed using an automated image analysis software (e.g.Perkin Elmer Technology) which allows the detection, quantification and segmentation of specific tissues through powerful pattern recognition algorithms. The machine-learning algorithm was typically previously trained to segment tumor from stroma and identify cells labelled.

A further object of the present invention relates to a method of treating cancer in a patient in need thereof comprising administering to the patient a therapeutically effective amount of a SK2 inhibitor.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxo sarcoma; liposarcoma; leiomyo sarcoma; rhabdomyo sarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In particular, the method of the present invention is particularly suitable for the treatment of cancer characterized by a low tumor infiltration of CTL and/or a high infiltration of MDSC.

Accordingly a further object of the present invention relates to a method of treating cancer in a patient in need thereof comprising i) quantifying the density of CTL in a tumor tissue sample obtained from the patient ii) comparing the density quantified at step i) with a predetermined reference value and iii) administering to the patient a therapeutically effective amount of the SK2 inhibitor when the density determined at step i) is lower than the predetermined value.

A further object of the present invention relates to a method of treating cancer in a patient in need thereof comprising i) quantifying the density of MDSC in a tumor tissue sample obtained from the patient ii) comparing the density quantified at step i) with a predetermined reference value and iii) administering to the patient a therapeutically effective amount of the SK2 inhibitor when the density determined at step i) is higher than the predetermined value.

Accordingly a further object of the present invention relates to a method of treating cancer in a patient in need thereof comprising i) quantifying the densities of CTL and MDSC in a tumor tissue sample obtained from the patient, ii) comparing the densities quantified at step i) with their corresponding predetermined reference value and iii) administering to the patient a therapeutically effective amount of the SK2 inhibitor when the density of CTL determined at step i) is lower than its corresponding predetermined value and the density of MDSC determined at step i) is higher than its corresponding predetermined value.

In some embodiments, the predetermined value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the density of CD8+ T cells in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DE SIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value correlates with the survival time of the patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”. In some embodiments, the predetermined reference value is determined by carrying out a method comprising the steps of a) providing a collection of tumor tissue samples from patient suffering from the cancer of interest; b) providing, for each tumor tissue sample provided at step a), information relating to the actual clinical outcome for the corresponding patient (i.e. the duration of the disease-free survival (DFS) and/or the overall survival (OS)); c) providing a serial of arbitrary quantification values; d) quantifying the density of CD8+ T cells for each tumor tissue sample contained in the collection provided at step a); e) classifying said tumor tissue samples in two groups for one specific arbitrary quantification value provided at step c), respectively: (i) a first group comprising tumor tissue samples that exhibit a quantification value for level that is lower than the said arbitrary quantification value contained in the said serial of quantification values; (ii) a second group comprising tumor tissue samples that exhibit a quantification value for said level that is higher than the said arbitrary quantification value contained in the said serial of quantification values; whereby two groups of tumor tissue samples are obtained for the said specific quantification value, wherein the tumor tissue samples of each group are separately enumerated; f) calculating the statistical significance between (i) the quantification value obtained at step e) and (ii) the actual clinical outcome of the patients from which tumor tissue samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g). For example, the density of CD8+ T cells has been assessed for 100 tumor tissue samples of 100 patients. The 100 samples are ranked according to the density of CD8+ T cells. Sample 1 has the highest density and sample 100 has the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the density of CD8+ T cells corresponding to the boundary between both subsets for which the p value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the density of CD8+ T cells with the range of values which are identified. In some embodiments, a cut-off value thus consists of a range of quantification values, e.g. centered on the quantification value for which the highest statistical significance value is found (e.g. generally the minimum p value which is found).

A further object of the present invention relates to a method of treating cancer in a patient in need thereof comprising administering to the patient a therapeutically effective combination of SK2 inhibitor with an immune checkpoint inhibitor, wherein administration of the combination results in enhanced therapeutic efficacy relative to the administration of the immune checkpoint inhibitor alone.

As used herein, the “immune checkpoint blockade therapy” relates to a therapy that consists in administering the patient with at least one immune checkpoint inhibitor.

As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitors includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.570, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1 MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immune checkpoint blockade therapy consists in administering to the patient a combination of a CTLA-4 antagonist and a PD-1 antagonist.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiments, the immune checkpoint inhibitor is selected from the group consisting of Ipilimumab, Nivolumab, Pembrolizumab, Atezolizuma, Avelumab, Durvalumab and Cemiplimab.

As used the terms “combination” and “combination therapy” are interchangeable and refer to treatments comprising the administration of at least two compounds administered simultaneously, separately or sequentially. As used herein the term “co-administering” as used herein means a process whereby the combination of at least two compounds is administered to the same patient. The at least two compounds may be administered simultaneously, at essentially the same time, or sequentially. The at least two compounds can be administered separately by means of different vehicles or composition. The at least two compounds can also be administered in the same vehicle or composition (e.g. pharmaceutical composition). The at least two compounds may be administered one or more times and the number of administrations of each component of the combination may be the same or different.

In some embodiments, the SK2 inhibitor of the present invention is particularly suitable for rendering a patient suffering from cancer eligible for an immune checkpoint blockade therapy.

The combination therapy may provide “synergy” and prove “synergistic”, i.e., the effect achieved when the active ingredients used together is greater than the sum of the effects that results from using the compounds separately.

In some embodiments, the patient is first administered with at least one cycle (C1) therapy with the SK2 inhibitor followed by administration of at least one cycle (C2) of immune checkpoint blockade therapy. As used herein, the term “cycle” refers to a period of time during the therapy is administered to the patient. Typically, in cancer therapy a cycle of therapy is followed by a rest period during which no treatment is given. Following the rest period, one or more further cycles of therapy may be administered, each followed by additional rest periods. In some embodiments, cycle (C1) comprises administering a dose of the SK2 inhibitor daily or every 2, 3, 4, or 5 days. In some embodiments, the SK2 inhibitor is administered continuously (i.e. every day) during cycle (C1). Typically cycle (C1) can last one or more days, but is usually one, two, three or four weeks long. In some embodiments cycle (C1) is repeated at least 2, 3, 4, 5, 6, 7, 8, 9 or 10 times before administering cycle (C2). In some embodiments, cycle (C2) consists in administering a dose of the immune checkpoint inhibitor weekly or every, 2, 4, or 5 weeks. In some embodiments, at the end of cycle (C1), the tumor infiltration of CD8+ T cells and/or MDSC is(are) quantified as described above. Then if the infiltration of CD8+ T cells increases and/or the infiltration of MDSC decreases after cycle (C1) then the patient is administered with cycle (C2). If the infiltration of CD8+ T cells and/or the infiltration of MDSC is not modified after the cycle (C1), the physician can decide to repeat cycle (C1).

A further object of the present invention relates to a method for enhancing the therapeutic efficacy of an immune checkpoint inhibitor administered to a patient as part of a treatment regimen, the method comprising administering to the patient a pharmaceutically effective amount of a SK2 inhibitor in combination with the immune checkpoint inhibitor.

As used herein, the expression “enhanced therapeutic efficacy,” relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy. As used herein, the expression “relative to” when used in the context of comparing the activity and/or efficacy of a combination composition comprising the immune checkpoint inhibitor with the SK2 inhibitor to the activity and/or efficacy of the immune checkpoint inhibitor alone, refers to a comparison using amounts known to be comparable according to one of skill in the art.

As used herein, the term “sphingosine kinase-2” or “SK2” refers to an enzyme that catalyzes the transformation of sphingosine to sphingosine-1-phosphate (S1P), i.e., phosphorylates sphingosine into SIP. Thus, as used herein the term “SK2 inhibitor” refers to any compound that is capable to inhibit SK2 expression or activity. As used herein the term ‘SK2 activity” refers to the production, release, expression, function, action, interaction or regulation of SK2, including, e.g., temporal, site or distribution aspects. The activity of SK2 includes modifications, e.g., covalent or non-covalent modifications of SK2 polypeptide, covalent or non-covalent modifications that SK2 induces on other substances, changes in the distribution of SK2 polypeptide, and changes that SK2 induces on the distribution of other substances. Any aspect of SK2 activity can be evaluated. Methods and techniques known to those skilled in the art. Examples of SK2 activity that can be evaluated include binding activity of SK2 polypeptide to a binding molecule; the effect of SK2 polypeptide on the posttranslational modification or stability of a target gene; the level of SK2 protein; the level of SK2 mRNA; or the level of SK2 modification, e.g., phosphorylation, acetylation, methylation, carboxylation or glycosylation. By binding molecule is meant any molecule to which SK2 can bind, e.g., a nucleic acid, e.g., a DNA regulatory region, a protein, a metabolite, a peptide mimetic, a non-peptide mimetic, an antibody, or any other type of ligand. Binding can be shown, e.g., by electrophoretic mobility shift analysis (EMSA), by the yeast or mammalian two-hybrid or three-hybrid assays, by competition with dimethylspingosine photoaffinity label or biotin-SK2 binding. Transactivation of a target gene by SK2 can be determined, e.g., in a transient transfection assay in which the promoter of the target gene is linked to a reporter gene, e.g., β-galactosidase or luciferase, and co-transfected with a SK2 expression vector. In some embodiments, the evaluations are done in vitro; in other embodiments the evaluations are done in vivo.

In some embodiments, the SK2 inhibitor of the present invention is a selective SK2 inhibitor. As used herein, the term “selective SK2 inhibitor” refers to a compound able to selectively inhibit SK2. In the context of the present invention, SK2 inhibitors are selective for SK2 as compared with SK1 (i.e. an enzyme that also catalyzes the transformation of sphingosine to sphingosine-1-phosphate). By “selective” it is meant that the affinity of inhibitor for the SK2 is at least 10-fold, preferably 25-fold, more preferably 100-fold and still preferably 300-fold higher than the affinity for SK1. The affinity of a compound may be quantified by measuring the activity of SK2 in the presence a range of concentrations of said inhibitor in order to establish a dose-response curve. Accordingly, a selective SK2 inhibitor is a compound for which the ratio Kd SK1/Kd SK2 is above 10:1, preferably 25:1, more preferably 100:1, still preferably 300:1.

SK2 inhibitors are well known in the art:
1: Sundaramoorthy P, Gasparetto C, Kang Y. The combination of a sphingosine kinase 2 inhibitor (ABC294640) and a Bcl-2 inhibitor (ABT-199) displays synergistic anti-myeloma effects in myeloma cells without a t(11;14) translocation. Cancer Med. 2018 May 15. doi: 10.1002/cam4.1543. [Epub ahead of print] PubMed PMID: 29761903; PubMed Central PMCID: PMC6051232.
2: Wallington-Beddoe C T, Bennett M K, Vandyke K, Davies L, Zebol J R, Moretti P A B, Pitman M R, Hewett D R, Zannettino A C W, Pitson S M. Sphingosine kinase 2 inhibition synergises with bortezomib to target myeloma by enhancing endoplasmic reticulum stress. Oncotarget. 2017 Jul. 4; 8(27):43602-43616. doi: 10.18632/oncotarget.17115. PubMed PMID: 28467788; PubMed Central PMCID: PMC5546428.
3: Britten C D, Garrett-Mayer E, Chin S H, Shirai K, Ogretmen B, Bentz T A, Brisendine A, Anderton K, Cusack S L, Maines L W, Zhuang Y, Smith C D, Thomas M B. A Phase I Study of ABC294640, a First-in-Class Sphingosine Kinase-2 Inhibitor, in Patients with Advanced Solid Tumors. Clin Cancer Res. 2017 Aug. 15; 23(16):4642-4650. doi: 10.1158/1078-0432.CCR-16-2363. Epub 2017 Apr. 18. PubMed PMID: 28420720; PubMed Central PMCID: PMC5559328.
4: Schrecengost R S, Keller S N, Schiewer M J, Knudsen K E, Smith C D. Downregulation of Critical Oncogenes by the Selective SK2 Inhibitor ABC294640 Hinders Prostate Cancer Progression. Mol Cancer Res. 2015 December; 13(12):1591-601. doi: 10.1158/1541-7786.MCR-14-0626. Epub 2015 Aug. 13. PubMed PMID: 26271487; PubMed Central PMCID: PMC4685021.
5: Gao P, Peterson Y K, Smith R A, Smith C D. Characterization of isoenzyme-selective inhibitors of human sphingosine kinases. PLoS One. 2012; 7(9):e44543. doi: 10.1371/journal.pone.0044543. Epub 2012 Sep. 10. PubMed PMID: 22970244; PubMed Central PMCID: PMC3438171.
6: Lim K G, Sun C, Bittman R, Pyne N J, Pyne S. (R)-FTY720 methyl ether is a specific sphingosine kinase 2 inhibitor: Effect on sphingosine kinase 2 expression in HEK 293 cells and actin rearrangement and survival of MCF-7 breast cancer cells. Cell Signal. 2011 October; 23(10):1590-5. doi:10.1016/j.cellsig.2011.05.010. Epub 2011 May 18. Erratum in: Cell Signal. 2012 June; 24(6):1115. PubMed PMID: 21620961; PubMed Central PMCID: PMC3148273.
7: Beljanski V, Lewis C S, Smith C D. Antitumor activity of sphingosine kinase 2 inhibitor ABC294640 and sorafenib in hepatocellular carcinoma xenografts. Cancer Biol Ther. 2011 Mar. 1; 11(5):524-34. Epub 2011 Mar. 1. PubMed PMID: 21258214; PubMed Central PMCID: PMC3087901.
8: French K J, Zhuang Y, Maines L W, Gao P, Wang W, Beljanski V, Upson J J, Green C L, Keller S N, Smith C D. Pharmacology and antitumor activity of ABC294640, a selective inhibitor of sphingosine kinase-2. J Pharmacol Exp Ther. 2010 April; 333(1):129-39. doi: 10.1124/jpet.109.163444. Epub 2010 January 8. PubMed PMID: 20061445; PubMed Central PMCID: PMC2846016.

In some embodiments, the SK2 inhibitor of the present invention has the formula (I)

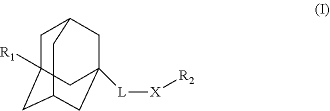

wherein
L is a bond or is C(R3,R4);
X is —C(R3,R4)N(R5)-, —C(O)N(R4)-, —N(R4)C(O)—, —C(R4,R5)-, —N(R4)-, —O—, —S—, —C(O)—, —S(O)2-, —S(O)2N(R4)- or —N(R4)S(O)2-;
R1 is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN. —NO2, —NH2, —CO2(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
R2 is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO2, —NH2, —CO2(alkyl). —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, mono or dialkylthiocarbamoyl, alkyl-S-alkyl, -heteroaryl-aryl, -alkyl-heteroaryl-aryl, —C(O)NH-aryl, -alkenyl-heteroaryl, —C(O)-heteroaryl, or -alkenyl-heteroaryl-aryl;
R3 is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO2, —NH2, —CO2(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above R1, R2, and R3 groups is optionally substituted with up to 5 groups that are independently (C1-C₆) alkyl, halogen, haloalkyl, —OC(O)(C1-C6 alkyl), —C(O)O(C1-C6 alkyl), —CONK′ R″. —OC(O)NR′ R″, —NR′ C(O)R″. —CF3, —OCF3, —OH, C1-C6 alkoxy, hydroxyalkyl, —CN, —CO2H, —SH, —S-alkyl, —SOR′ R″, —SO2R′, —NO2, or NR′ R″, wherein R′ and R″ are independently H or (C1-C6) alkyl, and wherein each alkyl portion of a substituent is optionally further substituted with 1, 2, or 3 groups independently selected from halogen, CN, OH, and NH2: and
R4 and R5 are independently H or alkyl, provided that when R3 and R4 are on the same carbon and R3 is oxo, then R4 is absent.

In some embodiments, the SK2 inhibitor of the present invention is selected from the group consisting of:
3-(4-Chloro-phenyl)-adamantane-1-carboxylic acidisopropylamide
3-(4-Chloro-phenyl)-adamantane-1-carboxylic acidcyclopropylamide
3-(4-Chloro-phenyl)-adamantane-1-carboxylic acid(2-ethyl sulfanyl-ethyl)-amide
3-(4-Chloro-phenyl)-adamantane-1-carboxylic acidphenylamide
Adamantane-1-carboxylic acid(4-hydroxy-phenyl)-amideNH
3-(4-Chloro-phenyl)-adamantane-1-carboxylic acid(4-hydroxy-phenyl)-amide
Acetic acid 4-{[3-(4-chloro-phenyl)-adamantane-1-carbonyl]-amino}-phenyl ester
3-(4-Chloro-phenyl)-adamantane-1-carboxylic acid(2,4-dihydroxy-phenyl)-amide
3-(4-Chloro-phenyl)-adamantane-1-carboxylic acid(3-hydroxymethyl-phenyl)-amide
Adamantane-1-carboxylic acid(4-cyanomethyl-phenyl)-amide
3-(4-Chloro-phenyl)-adamantane-1-carboxylic acid(4-cyanomethyl-phenyl)-amide
3-(4-Chloro-phenyl)-adamantane-1-carboxylic acidbenzylamide
3-(4-Chloro-phenyl)-adamantane-1-carboxylic acid4-tert-butyl-benzylamide
3-(4-Chloro-phenyl)-adamantane-1-carboxylic acid-4-methylsulfanyl-benzylamide
3-(4-Chloro-phenyl)-adamantane-1-carboxylic acid3-trifluoromethyl-benzamide
3-(4-Chloro-phenyl)-adamantane-1-carboxylic acid4-trifluoromethyl-benzylamide
3-(4-Chloro-phenyl)-adamantane-1-carboxylic acid3,5-bis-trifluoromethyl-benzylamide
3-(4-Chloro-phenyl)-adamantane-1-carboxylic acid3-fluoro-5-trifluoromethyl-benzylamide
3-(4-Chloro-phenyl)-adamantane-1-carboxylic acid2-fluoro-4-trifluoromethyl-benzamide
3-(4-Chloro-phenyl)-adamantane-1-carboxylic acid3, 5-di fluoro-benzylamide
3-(4-Chloro-phenyl)-adamantane-1-carboxylic acid3,4-difluoro-benzylamide
3-(4-Chloro-phenyl)-adamantane-1-carboxylic acid3,4, 5-trifluoro-benzylamide
3-(4-Chloro-phenyl)-adamantane-1-carboxylic acid3-chloro-4-fluoro-benzylamide
3-(4-Chloro-phenyl)-adamantane-1-carboxylic acid4-fluoro-3-trifluoromethyl-benzylamide
3-(4-Chloro-phenyl)-adamantane-1-carboxylic acid2-chloro-4-fluoro-benzylamide
3-(4-Chloro-phenyl)-adamantane-1-carboxylic acid(4-chloro-3-trifluoromethyl-benzylamide
3-(4-Chloro-phenyl)-adamantane-1-carboxylic acid3-aminomethyl-2,4,5,6-tetrachloro-benzylamide
3-(t-Chloro-phenyl)-adamantane-1-carboxylic acid [1-(4-chloro-phenyl)-ethyl]-amide
3-(4-Chloro-phenyl)-adamantane-1-carboxylic acid [1-(4-bromo-phenyl)-ethyl]-amide
3-(4-Chloro-phenyl)-adamantane-1-carboxylic acid4-methanesulfonyl-benzylamide
3-(4-Chloro-phenyl)-adamantane-1-carboxylic acid4-dimethylamino-benzylamide
3-(4-Chloro-phenyl)-adamantane-1-carboxylic acid4-trifluoromethoxy-benzylamide
3-(4-Chloro-phenyl)-adamantane-1-carboxylic acid3-trifluoromethoxy-benzylamide
3-(4-Chloro-phenyl)-adamantane-1-carboxylic acid4-phenoxy-benzylamide
Adamantane-1-carboxylic acid3,4-dihydroxy-benzylamide
3-(4-Chloro-phenyl)-adamantane-1-carboxylic acid3,4-dihydroxy-benzylamide
3-(4-Chloro-phenyl)-adamantane-1-carboxylic acidphenethyl-amide
3-(4-Chloro-phenyl)-admantane-1-carboxylic acid[2-(4-fluoro-phenyl)-ethyl]-amide
3-(4-Chloro-phenyl)-adamantane-1-carboxylic acid [2-(4-bromo-phenyl)-ethyl]-amide
3-(4-Chloro-phenyl)-adamantane-1-carboxylic acid [2-(4-hydroxy-phenyl)-ethyl]-amide
3-(4-Chloro-phenyl)-adamantane-1-carboxylic acid4-phenoxy-benzylamide
3-(4-Chloro-phenyl)-adamantane-1-carboxylic acid [2-(3-bromo-4-methoxy-phenyl)-ethyl]-amide
Adamantane-1-carboxylic acid[2-(3,4-dihydroxy-phenyl)-ethyl]-amide
3-(4-Chloro-phenyl)-adamantane-1-carboxylic acid [2-(3,4-dihydroxy-phenyl)-ethyl]-amide
3-(4-Chloro-phenyl)-adamantane-1-carboxylic acid(2-benzo[1,3]dioxol-5-yl-ethyl)-amide
3-(4-Chloro-phenyl)-adamantane-1-carboxylic acid [2-(3-phenoxy-phenyl)-ethyl]-amide
3-(4-Chloro-phenyl)-adamantane-1-carboxylic acid [2-(4-phenoxy-phenyl)-ethyl]-amide
3-(4-Chloro-phenyl)-adamantane-1-carboxylic acid(3-phenyl-propyl)-amide
3-(4-Chloro-phenyl)-adamantane-1-carboxylic acid(biphenyl-4-ylmethyl)-amide
Adamantane-1-carboxylic acid(1-methyl-piperidin-4-yl)-amide
3-(4-Chloro-phenyl)-adamantane-1-carboxylic acid(1-methyl-piperidin-4-yl)-amide
3-(4-Chloro-phenyl)-adamantane-1-carboxylic acid(4-methyl-piperazin-1-yl)-amide
3-(4-Chloro-phenyl)-adamantane-1-carboxylic acid(3-tert-butyl amino-propyl)-amide
3-(4-Chloro-phenyl)-adamantane-1-carboxylic acid(3-pyrrolidin-1-yl-propyl)-amide
3-(4-Chloro-phenyl)-adamantane-1-carboxylic acid [3-(2-oxo-pyrrolidin-1-yl)-propyl]-amide
Adamantane-1-carboxylic acid[2-(1-methyl-pyrrolidin-2-yl)-ethyl]-amide NH
3-(4-Chloro-phenyl)-adamantane-1-carboxylic acid[2-(1-methyl-pyrrolidin-2-yl)-ethyl]-amide
3-(4-Chloro-phenyl)-adamantane-1-carboxylic acid(2-morpholin-4-yl-ethyl)-amide
3-(4-Chloro-phenyl)-adamantane-1-carboxylic acid(2-piperazin-1-yl-ethyl)-amide
Adamantane-1-carboxylic acid(pyridin-4-ylmethyl)-amide
3-(4-Fluoro-phenyl)-adamantane-1-carboxylic acid(pyridin-4-ylmethyl)-amide
3-(4-Chloro-phenyl)-adamantane-1-carboxylic acid(pyridin-4-ylmethyl)-amide
Adamantane-1-carboxylic acid(pyridin-4-ylmethyl)-amide
3-(4-Chloro-phenyl)-adamantane-1-carboxylic acid(2-pyridin-4-yl-ethyl)-amide
3-(4-Chloro-phenyl)-adamantane-1-carboxylic acid(3-imidazol-1-yl-propyl)-amide
3-(4-Chloro-phenyl)-adamantane-1-carboxylic acid(2-methyl-1H-indol-5-yl)-amide
3-(4-Chloro-phenyl)-adamantane-1-carboxylic acid(1H-tetrazol-5-yl)-amide
3-(4-Chloro-phenyl)-adamantane-1-carboxylic acid(9-ethyl-9H-carbazol-3-yl)-amide
Adamantane-1-carboxylic acid[4-(4-chloro-phenyl)-thiazol-2-yl]-amide
3-(4-Chloro-phenyl)-adamantane-1-carboxylic acid [4-(4-chloro-phenyl)-thiazol-2-yl]-amide
3-(4-Chloro-phenyl)-adamantane-1-carboxylic acidbenzothiazol-2-yl amide
3-(4-Chloro-phenyl)-adamantane-1-carboxylic acid(5-chloro-benzooxazol-2-yl)-amide
3-(4-Chloro-phenyl)-adamantane-1-carboxylic acid(9H-purin-6-yl)-amide
[3-(4-Chloro-phenyl)-adamantan-1-ylmethyl]-isopropyl-amine
[3-(4-Chloro-phenyl)-adamantan-1-ylmethyl]-(4-trifluoromethyl-benzyl)-amine
[3-(4-Chloro-phenyl)-adamantan-1-ylmethyl]-(2-fluoro-4-trifluoromethyl-benzyl)-amine
[3-(4-Chloro-phenyl)-adamantan-1-ylmethyl]-(4-fluoro-3-trifluoromethyl-benzyl)-amine
[3-(4-Chloro-phenyl)-adamantan-1-ylmethyl]-(4-trifluoromethoxy-benzyl)-amine
[3-(4-Chloro-phenyl)-adamantan-1-yl methyl]-[2-(3-phenoxy-phenyl)-ethyl]-amine
[3-(4-Chloro-phenyl)-adamantan-1-ylmethyl]-(1-methyl-piperidin-4-yl)-amine
[3-(4-Chloro-phenyl)-adamantan-1-ylmethyl]-(4-methyl-piperazin-1-yl)-amine
N-tert-Butyl-N′-[3-(4-chloro-phenyl)-adamantan-1-yl methyl]-propane-1,3-diamine
[3-(4-Chloro-phenyl)-adamantan-1-ylmethyl]-(3-pyrrolidin-1-yl-propyl)-amine
[3-(4-Chloro-phenyl)-adamantan-1-ylmethyl]-[2-(1-methyl-pyrrolidin-2-yl)-ethyl]-amine
[3-(4-Chloro-phenyl)-adamantan-1-ylmethyl]-(2-morpholin-4-yl-ethyl)-amine
[3-(4-Chloro-phenyl)-adamantan-1-ylmethyl]-pyridin-4-ylmethyl-amine
[3-(4-Chloro-phenyl)-adamantan-1-ylmethyl]-(9-ethyl-9H-carbazol-3-yl)-amine
[3-(4-Chloro-phenyl)-adamantan-1-ylmethyl]-[5-(4-chloro-phenyl)-thiazol-2-yl]-amine
   1-[3-(4-Chloro-phenyl)-adamantan-1-yl]-ethylamine
{1-[3-(4-Chloro-phenyl)-adamantan-1-yl]-ethyl}-isopropyl-amine
Phenyl-[1-(3-phenyl-adamantan-1-yl)-ethyl]-amine
{1-[3-(4-Fluoro-phenyl)-adamantan-1-yl]ethyl}phenyl-amine
{1-[3-(4-Chloro-phenyl)-adamantan-1-yl]-ethyl}-phenyl-amine
(1-Adamantan-1-yl-ethyl)-benzyl-amine
Benzyl-[1-(3-phenyl-adamantan-1-yl)-ethyl]-amine
Benzyl-{1-[3-(4-fluoro-phenyl)-adamantan-1-yl]-ethyl}-amine
Benzyl-{1-[3-(4-chloro-phenyl)-adamantan-1-yl]ethyl}-amine
(4-tert-Butyl-benzyl)-{1-[3-(4-chloro-phenyl)-adamantan-1-yl]-ethyl}-amine
[1-(4-Bromo-phenyl)-ethyl]-{1-[3-(4-chloro-phenyl)-adamantan-1-yl]-ethyl}-amine
(1-Adamantan-1-yl-ethyl)-[2-(4-bromo-phenyl)-ethyl]-amine
[2-(4-Bromo-phenyl)-ethyl]-{1-[3-(4-chloro-phenyl)-adamantan-1-yl]-ethyl}-amine
(1-Adamantan-1-yl-ethyl)-(1-methyl-piperidin-4-yl)-amine
(1-Methyl-piperidin-4-yl)-[1-(3-phenyl-adamantan-1-yl)-ethyl]-amine
{1-[3-(4-Fluoro-phenyl)-adamantan-1-yl]-ethyl}-(1-methyl-piperidin-4-yl)-amine
{1-[3-(4-Chloro-phenyl)-adamantan-1-yl]-ethyl}-(1-methyl-piperidin-4-yl)-amine
{1-[3-(4-Chloro-phenyl)-adamantan-1-yl]-ethyl}-(4-methyl-piperazin-1-yl)-amine
{1-[3-(Phenyl)-adamantan-1-yl]-ethyl}-pyridin-4-ylmethyl-amine
{1-[3-(4-Chloro-phenyl)-adamantan-1-yl]-ethyl}-(6-chloro-pyridin-3-ylmethyl)-amine
{1-[3-(4-Chloro-phenyl)-adamantan-1-yl]-ethyl}-(2-pyridin-4-yl-ethyl)-amine
{1-[3-(4-Chloro-phenyl)-adamantan-1-yl]-ethyl}-(3H-imidazol-4-ylmethyl)-amine
{1-[3-(4-Chloro-phenyl)-adamantan-1-yl]-ethyl}-(2-methyl-1H-indol-5-yl)-amine
{1-[3-(4-Chloro-phenyl)-adamantan-1-yl]-ethyl}-(9-ethyl-9H-carbazol-3-yl)-amine
{1-[3-(4-Chloro-phenyl)-adamantan-1-yl]-ethyl}-(9-ethyl-9H-carbazol-3-ylmethyl)-amine
9-Ethyl-9H-carbazole-3-carboxylic acid {1-[3-(4-chloro-phenyl)-adamantan-1-yl]-ethyl}-amide
1-{1-[3-(4-Chloro-phenyl)-adamantan-1-yl]-ethyl}-3-(4-chloro-3-trifluoromethyl-phenyl)-urea
1-{1-[3-(4-Chloro-phenyl)-adamantan-1-yl]-ethyl}-3-(4-chloro-3-trifluoromethyl-phenyl)-urea NH CH3
(4-Bromo-thiophen-2-ylmethyl)-{1-[3-(4-chloro-phenyl)-adamantan-1-yl]-ethyl}-amine
{1-[3-(4-Chloro-phenyl)-adamantan-1-yl]-ethyl}-(4-phenyl-thiophen-2-ylmethyl)-amine
3-Phenyl-adamantane-1-carboxylicacid
3-(4-Fluoro-phenyl)adamantane-1-carboxylic acid
3-(4-Chloro-phenyl)adamantane-1-carboxylic acid
1-Adamantan-1-yl-ethanone
1-(3-Phenyl-adamantan-1-yl)-ethanone
1-[3-(4-Fluoro-phenyl)adamantan-1-yl]-ethanone
1-[3-(4-Chloro-phenyl)adamantan-1-yl]-ethanone
2-(Adamantane-1-carbonyl)-malonicacid dimethyl ester H
2-[3-(4-Chloro-phenyl)adamantane-1-carbonyl]-malonic acid dimethyl ester
3-(4-Chloro-phenyl)-1-[3-(4-chloro-phenyl)-adamantan-1-yl]-propenone
4-{3-[3-(4-Chloro-phenyl)-adamantan-1-yl]-3-oxo-propenyl}-benzonitrile
1-[3-(4-Chloro-phenyl)adamantan-1-yl]-3-(4-hydroxy-phenyl)-prop enone
1-[3-94-Chloro-phenyl)-adamantan-1-yl]-3-naphthalen-2-yl-prop enone
1-[3-(4-Chloro-phenyl)-adamantan-1-yl]-3-(6-chloro-pyridin-3-yl)-propenone
1-[3-(4-Chloro-phenyl)-adamantan-1-yl]-3-(1H-imidazol-4-yl)-prop enone
1-[3-(4-Chloro-phenyl)-adamantan-1-yl]-3-(9-ethyl-9H-carbazol-3-yl)-propanone, and
1-[3-(4-Chloro-phenyl)-adamantan-1-yl]-3-(4-phenyl-thiophen-2-yl)-propenone

In some embodiments, the SK2 inhibitor is selected from the group consisting of:
acetic acid 2-acetoxy-5-(2-{[3-(4-chlorophenyl)-adamantane-1-carbonyl]-amino}ethyl)phenyl ester;
propionic acid 2-propionyloxy-5-(2-{[3-(4-chlorophenyl)-adamantane-1-carbonyl]-amino}ethyl)phenyl ester;
butyric acid 2-butyryloxy-5-(2-{[3-(4-chlorophenyl)-adamantane-1-carbonyl]-amino}ethyl)phenyl ester;
isobutyric acid 5-(2-{[3-(4-chlorophenyl)adamantane-1-carbonyl]amino}ethyl)-2-hydroxyphenyl ester; and
[2-Amino-3-methyl-butyric acid 5-(2-{[3-(4-chlorophenyl)adamantane-1-carbonyl]amino}ethyl)-2-hydroxyphenyl ester.

In some embodiments, the SK2 inhibitor is ABC294640 [3-(4-chlorophenyl)-adamantane-1-carboxylic acid (pyridin-4-ylmethyl)amide].

In some embodiments, the SK2 inhibitor is an inhibitor of SK2 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of SK2 mRNA by binding thereto and thus preventing protein translation or increasing mRNA degradation, thus decreasing the level of SK2, and thus activity, in a cell. For example, antisense oligonucleotides of at least about 15 bases and complementary to unique regions of the mRNA transcript sequence encoding SK2 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. SK2 gene expression can be reduced by contacting a subject or cell with a small double stranded RNA (dsRNA), or a vector or construct causing the production of a small double stranded RNA, such that SK2 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing SK2. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of the antibody or antibody portion are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of an inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled inhibitor of the present invention, fragment or mini-antibody derived from the inhibitor of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

According to the present invention, the active agent (i.e. SK2 inhibitor or immune checkpoint inhibitor) is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1. Genetic deletion of SphK2 leads to a significant delay in the melanoma tumor growth and burden, enhancing the entry of tumor-infiltrating effector lymphocytes in immunocompetent mice.

8-10-week-old Sphk2 or Sphk2^{+/+} female mice were orthotopically grafted with YUMM1.7 murine melanoma cell line (300.10³ cells), tumor volume was monitored over time and tumor weight was measured at day 10 post-tumor injection. (A) Growth curves are presented as mean of tumor volume ±SEM for each depicted day post-tumor injection and are representative of at least two independent experiments (n=6-8 mice per group). (B) Tumor weight graph shows in milligrams (mg) the differences observed at day 10 after tumor inoculation. (C-D) Immune infiltrate within the tumor was analyzed at day 10 post-tumor injection for lymphoid lineage-derived populations (C) and myeloid lineage-derived populations (D) by flow cytometry. Frequencies of CD8α⁺ T cells, regulatory CD4⁺ T cells (Tregs), PD-1⁺-expressing CD8α⁺ T cells, and CD8α⁺/Tregs ratio; and PD-1 MFI are represented (C). Frequencies of neutrophils and polymorphonuclear-MDSCs; and CD8α⁺/MDSCs ratio are represented (D). Each symbol represents an independent tumor (n=6-8 mice per group). Graphs are representative of two pooled independent experiments. (A) Growth curves were compared using repeated measures (RM) two-way ANOVA/Sidak's test. (B) Tumor weights were compared using Mann-Whitney test. Frequencies data were compared using Mann-Whitney test (C and D). *p<0.05, **p<0.01, ***p<0.001, ****p<0.0001.

FIG. 2. Combination of SphK2 deficiency with immune-checkpoint blockade leads to tumor rejection, increases survival rate and induces potent vaccination.

8-10-week-old Sphk2 or Sphk2^{+/+} female mice were orthotopically grafted with YUMM1.7 murine melanoma cell line (300.10³ cells), then treated or not by immunotherapy and tumor volume and survival rate were monitored and estimated over time (n=4-5 mice per group). Tumor volumes are presented as mean of tumor volume ±SEM for each depicted day post-tumor injection. (A) Mice received a combo treatment of anti-PD-1/anti-CTLA-4 or isotype control at days 5, 8 and 12 post-tumor injection. (C) Mice were treated with anti-PD-1 or isotype control at days 5, 8 and 12 post-tumor injection. (E, F) Sphk2 mice treated with combo or only anti-PD-1 were re-challenged around 90 days post-tumor injection at the same site of primary injection with YUMM1.7 cells (1.10⁶ cells). (A, C) Growth curves were compared using repeated measure (RM) two-way ANOVA/Sidak's test. (B, D) Cumulative survival curves were analyzed using Log-rank (Mantel-Cox) test. *p<0.05, **p<0.01, ***p<0.001, and ****p<0.0001.

FIG. 3. SphK2-deficient CD8α⁺ T cells are the key immune regulators in the control of tumor development.

8-10-week-old Sphk2 or Sphk2^{+/+} female mice were orthotopically grafted with YUMM1.7 murine melanoma cell line (300.10³ cells), then treated or not with a depleting monoclonal CD8a antibody and tumor volume was monitored and estimated over time (n=6 mice per group). (A, B) Tumor volumes are presented as mean of tumor volume ±SEM for each depicted day post-tumor injection. Mice received a treatment of anti-CD8a or isotype control 2 days prior to tumor injection, then at days 2, 4, 6, 8, 10 and 12 post-tumor injection. (A) Growth curves were compared using repeated measure (RM) two-way ANOVA/Sidak's test. *p<0.05, **p<0.01, ***p<0.001, and ****p<0.0001.

FIG. 4. SphK2-deficient Polymorphonuclear-MDSCs are critical mediators implicated in loss of tumor growth control.

8-10-week-old Sphk2 or Sphk2^{+/+} female mice were orthotopically grafted with YUMM1.7 murine melanoma cell line (300.10³ cells), then treated or not with a depleting monoclonal Ly6G antibody and tumor volume was monitored and estimated over time (n=5-6 mice per group). Growth curves are representative of two pooled independent experiments. (A, B) Tumor volumes are presented as mean of tumor volume ±SEM for each depicted day post-tumor injection. Mice received a treatment of anti-Ly6G or isotype control from palpable tumor (day 5), then at days 7, 9, 11, 13 and 15 post-tumor injection. (A) Growth curves were compared using repeated measure (RM) two-way ANOVA/Sidak's test. *p<0.05, **p<0.01, ***p<0.001, and ****p<0.0001.

Results:

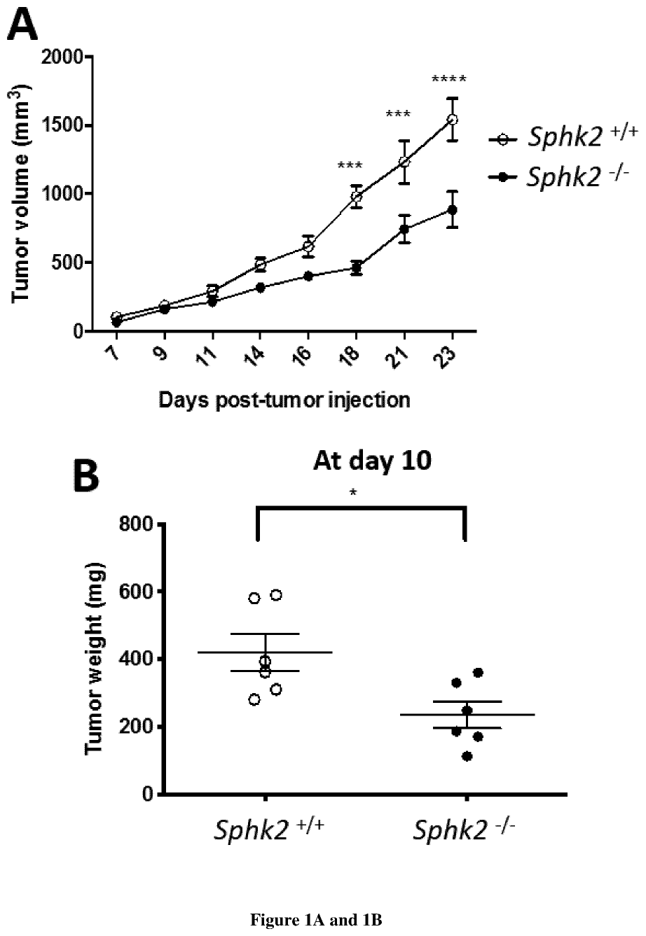

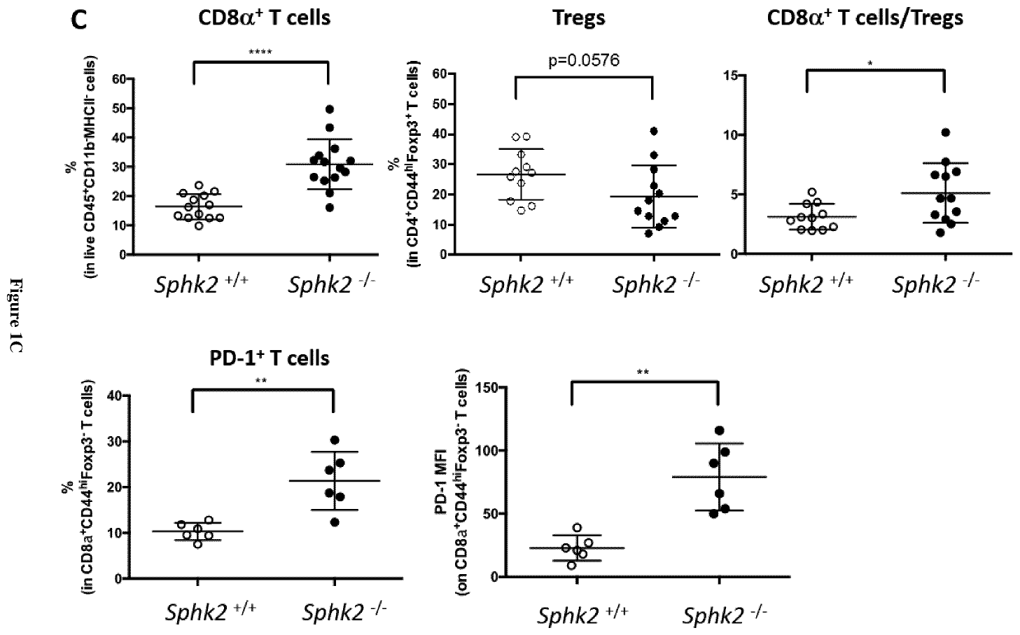

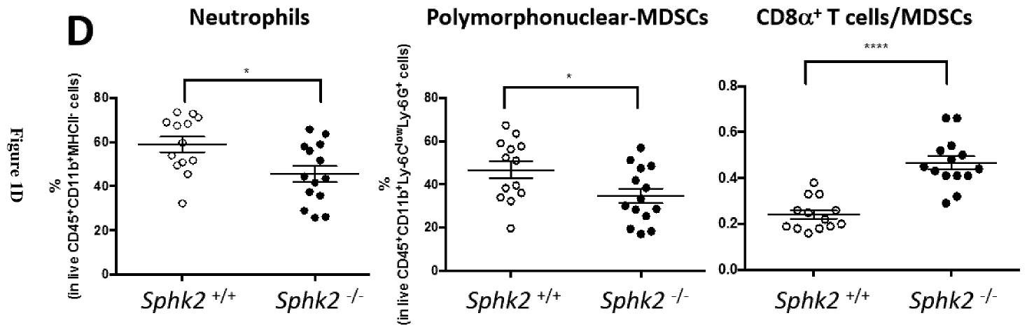

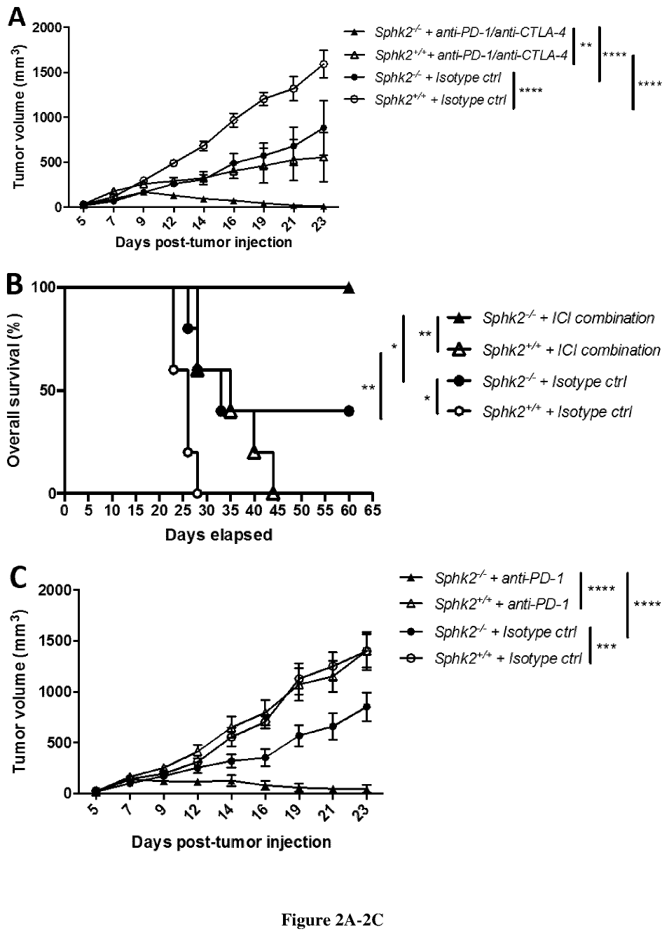

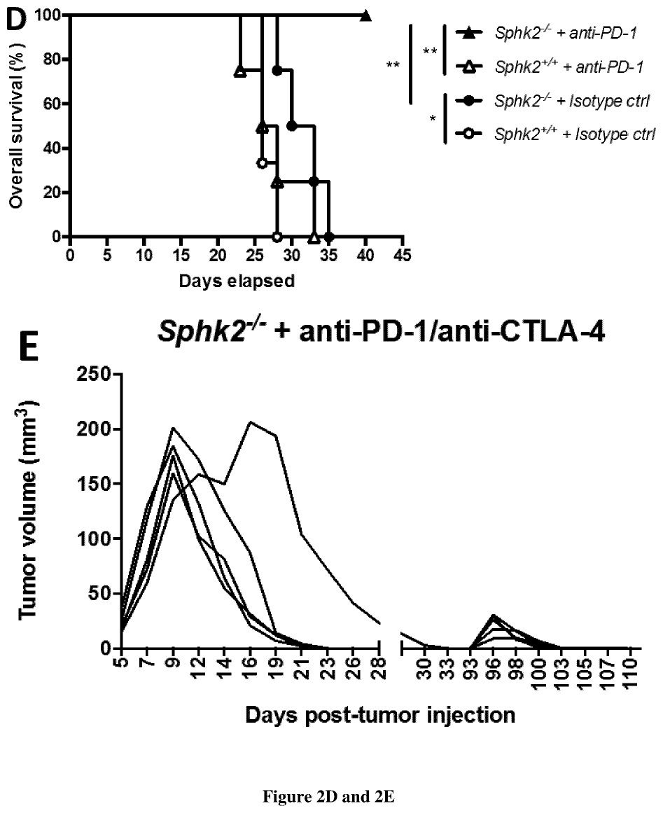

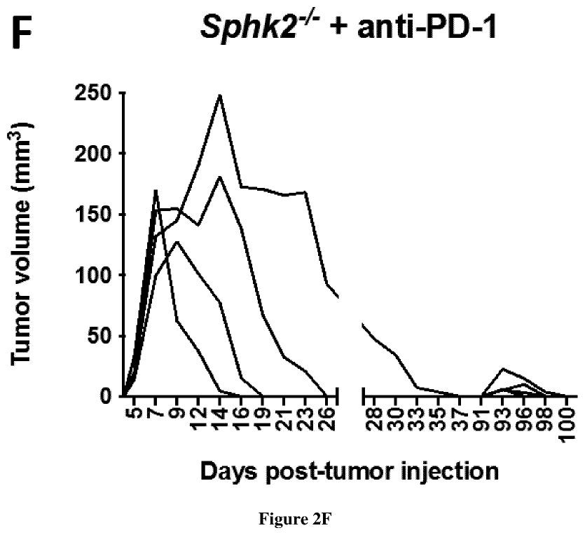

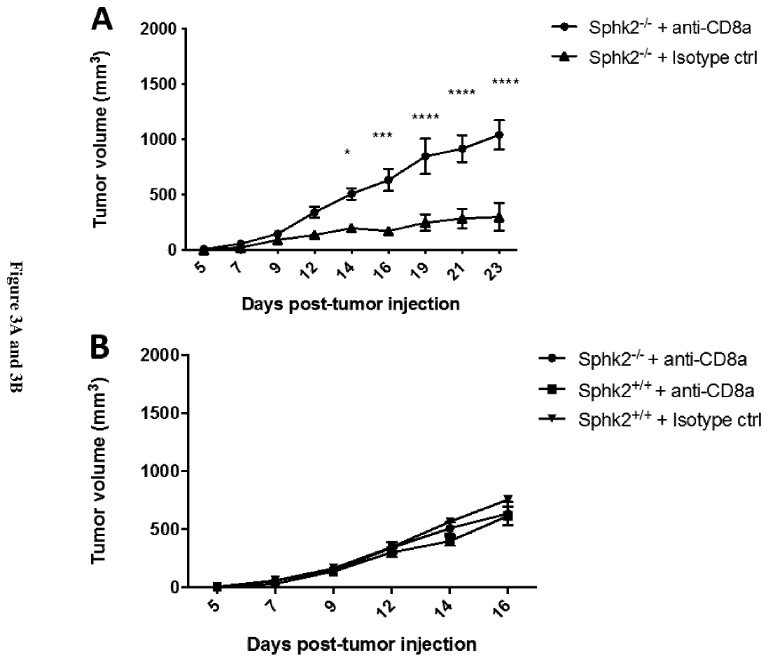

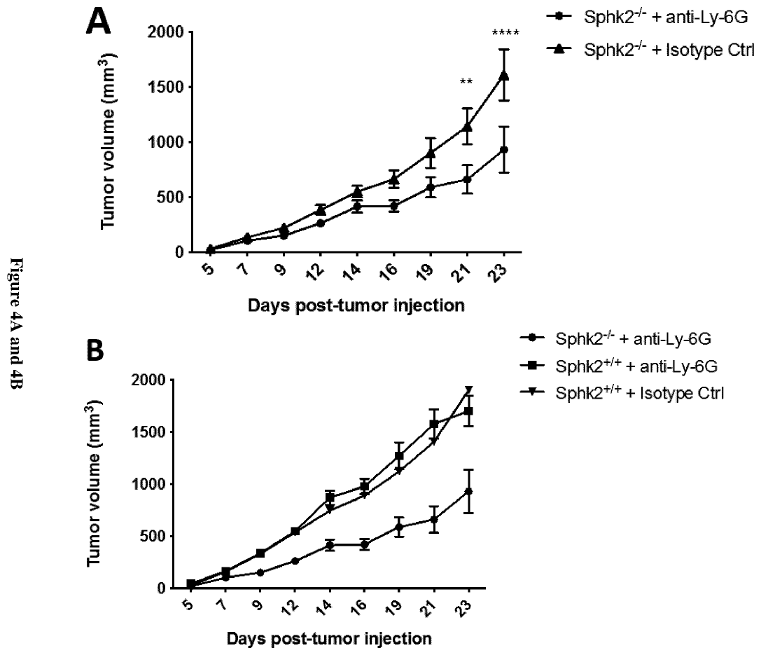

Immune checkpoint blockade therapy is based on the inhibition of the tumor-mediated suppression of anticancer immune responses. However, the efficacy and effectiveness of said therapy vary greatly across individual patients and among different tumor types. A substantial unmet need is thus to identify novel targets that can enhance the therapeutic efficacy of the immune checkpoint blockade therapy. S1P is produced by sphingosine kinases (i.e. SK1 and SK2) that catalyze the phosphorylation of sphingosine to SIP. SK2 inhibitors were described as suitable for the treatment of cancer. However, the role of SK2 in the immune tumor microenvironment has never been investigated. The inventors now showed that genetic deletion of SPHK2 leads to a delay in the melanoma tumor growth and burden (FIGS. 1A and 1B), enhancing the entry of tumor-infiltrating effector lymphocytes (FIG. 1C). In particular the increase of tumor-infiltrating effector lymphocytes in the tumor is associated with a decrease in the amount of tumor-infiltrating myeloid-derived suppressor cells (FIG. 1D). Moreover, the combination of SPHK2 deficiency with immune-checkpoint blockade leads to tumor rejection, increases survival rate and induces potent vaccination (FIGS. 2A, 2B, 2C, 2D, 2E and 2F). In addition, SphK2-deficient CD8α⁺ T cells have been shown as the key immune regulators in the control of tumor development (FIGS. 3A and 3B). Conversely, SphK2-deficient Polymorphonuclear-MDSCs have been shown as critical mediators implicated in loss of tumor growth control (FIGS. 4A and 4B). Accordingly, the present invention relates to use of SK2 inhibitors in combination with immune checkpoint blockade therapy for the treatment of cancer.

### REFERENCES

Throughout this application, various references describe the state of the art to which this invention pertains. The disclosures of these references are hereby incorporated by reference into the present disclosure.

1. A method of increasing the amount of tumor infiltrating cytotoxic T lymphocytes and/or reducing the amount of tumor-infiltrating myeloid-derived suppressor cells in a patient suffering from cancer comprising administering to the patient a therapeutically effective amount of a SK2 inhibitor.

2. (canceled)

3. A method of treating cancer in a patient in need thereof comprising
i) quantifying the density of cytotoxic T lymphocytes (CTL) in a tumor tissue sample obtained from the patient and ii) administering to the patient a therapeutically effective amount of an SK2 inhibitor when the density determined at step i) is lower than a corresponding predetermined reference value; and/or iii) quantifying the density of myeloid-derived suppressor cells (MDSC) in the tumor tissue sample obtained from the patient and iv) administering to the patient a therapeutically effective amount of the SK2 inhibitor when the density determined at step iii) is higher than a corresponding predetermined reference value.

4. (canceled)

5. The method of claim 3 comprising i) quantifying the densities of both CTL and MDSC inn the tumor tissue sample obtained from the patient, and ii) administering to the patient a therapeutically effective amount of the SK2 inhibitor when the density of CTL determined at step i) is lower than its corresponding predetermined value and the density of MDSC determined at step i) is higher than its corresponding predetermined value.

6. A The method of claim 3 further comprising administering to the patient, in combination with the SK2 inhibitor, a therapeutically effective combination of SK2 inhibitor with amount of an immune checkpoint inhibitor, wherein administration of the combination results in enhanced therapeutic efficacy relative to the administration of the immune checkpoint inhibitor alone.

7. The method of claim 6 wherein the patient is first administered with at least one cycle (C1) therapy with the SK2 inhibitor followed by administration of at least one cycle (C2) of immune checkpoint blockade therapy.

8. A method for enhancing the therapeutic efficacy of an immune checkpoint inhibitor administered to a patient as part of a treatment regimen, the method comprising administering to the patient a pharmaceutically effective amount of a SK2 inhibitor in combination with the immune checkpoint inhibitor.

9. The method of claim 6 wherein the immune checkpoint inhibitor is selected from the group consisting of PD-1 antagonists, PD-L1 antagonists, PD-L2 antagonists, CTLA-4 antagonists, VISTA antagonists, TIM-3 antagonists, LAG-3 antagonists, IDO antagonists, KIR2D antagonists, A2AR antagonists, B7-H3 antagonists, B7-H4 antagonists, and BTLA antagonists.

10. The method of claim 6 wherein the immune checkpoint inhibitor is selected from the group consisting of Ipilimumab, Nivolumab, Pembrolizumab, Atezolizuma, Avelumab, Durvalumab and Cemiplimab.

11. The method according to claim 1 wherein the SK2 inhibitor is a selective SK2 inhibitor.

12. The method of claim 11 wherein the selective SK2 inhibitor is ABC294640.

13. The method according to claim 1 wherein the SK2 inhibitor is an inhibitor of SK2 expression.
